(19) United States
(12) Patent Application Publication
(10) Pub. No.: US 2007/0083263 A1
(43) Pub. Date: Apr. 12, 2007

(54) Ossiclse prosthesis with elastic rotary joint

(76) Inventors: Uwe Steinhardt, Hirrlingen (DE); Heinz Kurz, Dusslingen (DE)

Correspondence Address: STRIKER, STRIKER & STENBY, 103 EAST NECK ROAD, HUNTINGTON, NY, 11743, US

(21) Appl. No.: 11/544,814

(22) Filed: Oct. 6, 2006

(30) Foreign Application Priority Data
Oct. 11, 2005 (DE) 10 2005 048 618.5

(51) Int. Cl. A61F 2/18

(52) U.S. Cl. 623/10

(57) ABSTRACT

An ossicle prothesis (10) which includes, at one end, a first fastening element (11) designed as a top plate for mechanical connection with the tympanic membrane and, at the other end, a second fastening element (12) for mechanical connection with a component of the ossicular chain or with the inner ear; it also includes a rotary joint between the two fastening elements, which includes a receiving part (13) in which a rotary element (14)—which is fixedly connected with a longitudinal shank (16) which connects the two fastening elements with each other—is hingedly supported, is characterized by the fact that the receiving part is made of an elastic plastic, the receiving part is rigidly connected with the first fastening element, and the receiving part includes a cavity which is shaped such that, in the installed state, it encloses at least half of the surface of the rotary element. This results in an articulation point which is easy and cost-effective to manufacture and provides the necessary post-operative flexibility and variability of the prosthesis while greatly improving the quality of sound conduction through the prosthesis.

The present invention relates to an ossicle prosthesis which replaces or bridges at least one component or parts of a component of the ossicular chain. The ossicles prosthesis includes, at one end, a first fastening element designed as a top plate for mechanical connection with the tympanic membrane and, at the other end, a second fastening element for mechanical connection with a component or parts of the ossicular chain or with the inner ear; it also includes a rotary joint between the two fastening elements. The rotary joint includes receiving part in which a rotary element is hingedly supported, the rotary element being fixedly connected with a longitudinal shank which connects the two fastening elements with each other.

A device of this type is made known in DE 20 2005 003 782 U1.

Ossicle prostheses are used in cases in which the ossicles of the human middle ear are missing or damaged, either entirely or partially, to conduct sound from the tympanic membrane to the inner ear. The ossicles prosthesis includes two ends. Depending on the specific circumstances, one end of the ossicles prosthesis is fastened to the limb of incus of the human ossicular chain, e.g., using a top plate, and the other end of the ossicle prosthesis is fastened, e.g., to the stapes of the human ossicular chain, or it is inserted directly into the inner ear. In many cases, with the known ossicle prostheses, sound conduction between the tympanic membrane and the inner ear is limited, because these known ossicle prostheses do not fully replace the natural anatomical formations of the ossicular chain.

After the prosthesis has been surgically implanted in the middle ear and the tympanic membrane has been closed, the recovery phase begins. Scars form during this period, and they produce unforeseeable forces, which can cause the prosthesis to move out of its local position. When there is a stiff connection between the top plate and the shank, increased pressure peaks can result between the edge of the top plate and the tympanic membrane, or the transplant between the tympanic membrane and the top plate. These pressure peaks can be so high that they could penetrate the tympanic membrane. For this reason, it is very helpful when the prosthesis has a certain amount of post-surgical mobility, so that the top plate can automatically adapt, post-operatively, to the position of the tympanic membrane.

Since, in addition, the unique anatomical features of the ear, such as the position, shape and size of the stapes, incus, hammer and tympanic membrane vary, it is very advantageous when ossicle prostheses are not designed to be rigid, but rather that they have a certain amount of flexibility or variability.

To attain this flexibility/variability, it is known to use various fastening and attachment devices for ossicles, which have elastic parts and/or joints. A hinged connection of this type between a fastening element which can be installed on the base of stapes and the longitudinal shank is described, e.g., in EP 1 181 907 B1, and is offered by the applicant under the trade mark “Ball-Joint”.

Given the considerable variation due to anatomy and genetics, middle ear prostheses having different lengths are required in otology for reconstruction of the ossicular chain as part of tympanoplasty. At this time, however, it is not possible to determine exactly what length is required before surgery is performed. For this reason, one either has to stockpile an extensive variety of protheses having different lengths, which is expensive, or the lengths of the prostheses must be variable, so that they can be matched to the particular patient immediately before they are implanted. Since the relative position of the ossicle prosthesis relative to the tympanic membrane differs slightly from person to person, the middle ear must be measured exactly in terms of the position of the fastening elements before the prosthesis is inserted. The required shank length cannot be determined until surgery is underway, however, which means that a set of prostheses having different lengths or one prosthesis with a variable shank length must be provided every time surgery is performed.

A favorable solution, with which the length of the shank can be trimmed to the particular length with minimal fabrication expenditure for the shank and, therefore, more cost-effectively, is described, e.g., in EP 0 998 884 B1. Ossicle prostheses of this type are offered by the applicant under the trade mark “VARIO”. With these known prostheses, the shank extends through a through-hole in the first fastening element, which is designed as a top plate. The shank can be axially displaced through the through-hole such that it extends beyond the outside of the top plate and can be trimmed there; the through-hole can be constricted afterward in order to fix the shank in position at the desired point.

A similar trimming technique is also described in U.S. Pat. No. 6,168,625 B1 and DE 100 45 158 A1. In this case, the shank—which is manufactured as a type of shaft—has a large number of notches along its axis, which are designed to serve as intended breaking points for the subsequent, unproblematic trimming step.

However, the latter ossicle prostheses do not include the articulation points described above for producing post-surgical mobility. In contrast, the ossicle prosthesis described in DE 20 2005 003 782 U1 cited initially—with which the shank includes a large number of balls which abut each other, form a ball chain and can be easily shortened to the desired length during surgery—combines the advantages of both types of prostheses.

The disadvantage of this known ossicle prosthesis is the type and design of the rotary joint. In this case, the receiving part for the rotary element is composed of two metallic segments which extend in parallel and enclose a gap-forming space between them, in which a ball which is part of the longitudinal shank is hingedly supported in two circular recesses in the segments. The two metallic segments extend into an opening in the first fastening element and are laterally movable and bendable so that, when the rotary joint is installed, the shank can be deflected in an undefined manner transversely to its axial direction, and a good, hard connection between the first and second fastening element is not ensured, which also means that optimal sound conduction through the prosthesis is not ensured.

The object of the present invention, therefore, is to improve a generic device of the type described initially such that an articulation point which is easy and cost-effective to manufacture is provided in order to attain the necessary post-operative flexibility and variability of the prosthesis while also greatly improving the quality of sound conduction through the prosthesis.

According to the present invention, this object is attained in a surprisingly simple and effective manner by manufacturing the receiving part using an elastic plastic and connecting it rigidly with the first fastening element, and by the fact that the receiving part includes a cavity which is shaped such that, in the installed state, it encloses at least half of the surface of the rotary element.

As a result, the advantages of the known ossicle prosthesis described above according to DE 20 2005 003 782 U1—which defines the species—are easily utilized, but the relatively hard, inventive design of the rotary joint enables optimal sound conduction through the prosthesis. This is ensured by the fact that—in contrast to the ball joint according to DE 20 2005 003 782 U1—the receiving part surrounds the rotary element fixedly and relatively rigidly in a large surface region. In some embodiments of the present invention, the enclosed region can encompass slightly less than half of the surface of the rotary element, while in other embodiments, it can encompass more than half of the surface of the rotary element. The use of elastic plastic for the receiving part ensures that the desired post-operative mobility is provided nevertheless, and it simplifies installation of the rotary elements. The special geometry of the receiving part and the rotary element after installation prevents the configuration from slipping in an undefined manner and permits swivel joint motions to take place only to a specifiable extent.

It is also very important that an automatic force adjustment take place post-operatively in the swivel joint support. It must not be too high, or no motion will take place. It should not be too low, either, because this could result in erroneous signal transmission. An optimal solution for this is also provided via the inventive device.

In this context, an embodiment of the present invention is particularly advantageous with which the elastic material of the receiving part is selected such that its surface has good antifrictional properties at room temperature. This easily allows disturbing frictional influences in the joint motion to be minimized.

In an embodiment which is relatively easy to manufacture, the rotary element is designed as a sphere or part of a sphere, particularly as a hemisphere.

According to an advantageous refinement of this embodiment, the cavity in the receiving part has the shape of a spherical shell which matches the rotary element.

According to an alternative embodiment of the present invention, the rotary element is designed as an ellipsoid of revolution or part of an ellipsoid of revolution. Although this requires slightly more manufacturing expenditure than a ball joint part, it provides advantages in terms of joint mobility.

The two embodiments described can be refined in that the rotary element engages in a cavity designed as a conical opening of the receiving part. This is also technically particularly easy to realize.

An alternative refinement is characterized by the fact that the cavity of the receiving part is cylindrical and, when the prosthesis is installed, the cavity presses against the rotary element slightly.

A further preferred embodiment of the present invention relates to an ossicle prosthesis with which the receiving part is inserted into and, particularly, is pressed into the first fastening element.

As an alternative to or in addition to attaining a particulary secure hold, according to other embodiments of the inventive ossicle prosthesis, the receiving part can be bonded or welded together with the first fastening element.

An alternative embodiment of the inventive ossicle prosthesis with which the receiving part is an integral component of the first fastening element—which is designed as a top plate—is particularly compact and therefore economical to manufacture.

With preferred embodiments, the receiving part of the inventive ossicle prosthesis is made of silicone or PTFE, and the rotary element and the longitudinal shank are made of metal.

In terms of particularly high post-operative mobility of the inventive ossicle prosthesis, an embodiment is particularly advantageous with which the longitudinal shank includes a large number of further rotary elements which abut each other.

In a refinement of this embodiment which is particularly easy and economical to manufacture, the shape of the adjoining, further rotary elements of the longitudinal shank is identical to that of the rotary element—they have the same outer diameter in particular—and they are located equidistantly along the axis of the shank. This also simplifies handling when the shank is cut to size during surgical implantation.

The prosthesis will be designed according to the particular defect to be eliminated or at least ameliorated in terms of its effect on the patient via use of the inventive ossicle prosthesis. With all embodiments of the present invention, the first fastening element will include a top plate designed to rest on the tympanic membrane.

A class of embodiments of the inventive device is characterized by the fact that the second fastening element is designed in the shape of a plate, a bell, a sleeve or a punch, or it is designed as a clip.

In refinements of these embodiments, the ossicle prosthesis is fastened via the top plate to the tympanic membrane and via the second fastening element to the incus or stapes.

Alternative embodiments can provide that the ossicle prosthesis is coupled via its end on which the second fastening element is mounted directly to the inner ear—using a plunger, in particular—by opening up the human cochlea (=cochleotomy).

According to a preferred embodiment of the present invention, the prosthesis or parts thereof are made of biocompatible plastics, particularly silicone, or fibrous composite materials. Post-operative rejection reactions can be prevented in most cases as a result.

The inventive ossicle prosthesis or parts thereof can be made of titanium and/or gold and/or tantalum and/or an alloy of these metals.

In terms of the post-operative position adjustment described above, embodiments of the present invention are advantageous with which the prosthesis or parts thereof are made of a material with memory effect, Nitinol in particular.

According to an very particularly preferred embodiment of the inventive device, the mass distribution of the individual parts of the prosthesis is calculated depending on a desired, specifiable frequency response of sound conduction in the middle ear. This allows the sound propagation properties to be tuned to a certain extent using a custom-made ossicle prosthesis.

With special embodiments, a tuning effect of this type can be attained, e.g., by fastening at least one additional mass to a part of the ossicular chain or the prosthesis depending on a desired, specifiable response of sound conduction in the middle ear.

With advantageous refinements of these embodiments, the additional mass is fastened to a part of the ossicular chain or the prosthesis using a clip.

Finally, a further embodiment of the present invention is characterized by the fact that the prosthesis is connected with an active vibrating part of a hearing aid which is active and implantable, in particular. This also enables further hearing damage to be prevented or at least ameliorated in terms of its effect by using modern electronics.

Further features and advantages of the present invention result from the detailed description of exemplary embodiments of the invention presented below with reference to the figures in the drawing, which shows the details that are essential to the present invention. Further features and advantages of the present invention also result from the claims. The individual features can be realized individually, or they can be combined in any possible manner in different variations of the present invention.

Exemplary embodiments of the present invention are depicted in the schematic drawing and are described in greater detail in the description below.

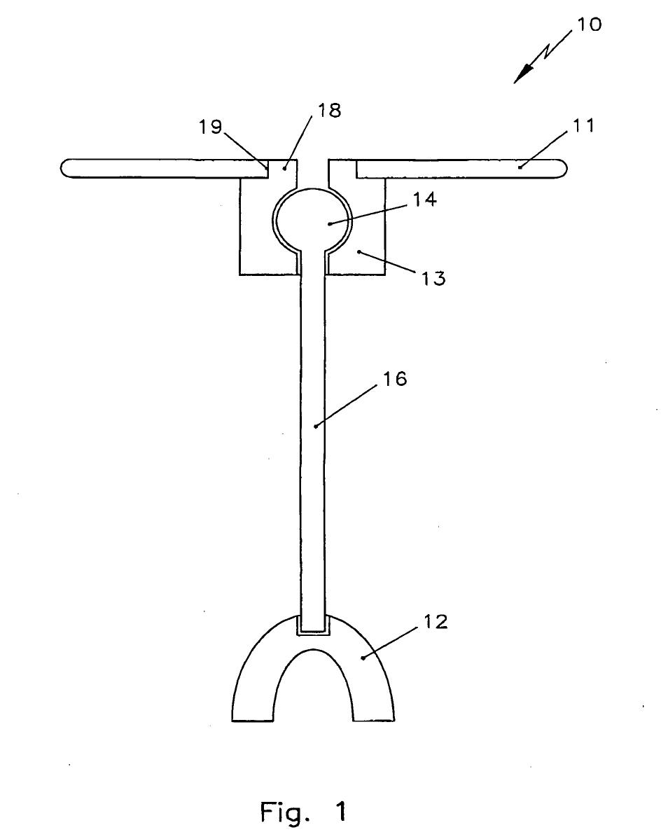

FIG. 1 shows a schematic longitudinal cross-section through an embodiment of the inventive ossicle prosthesis with a first fastening element designed as a top plate, and a bell-shaped second fastening element on the other end of the longitudinal shank, and with a spherical rotary element which is surrounded by a receiving part designed as a spherical shell;

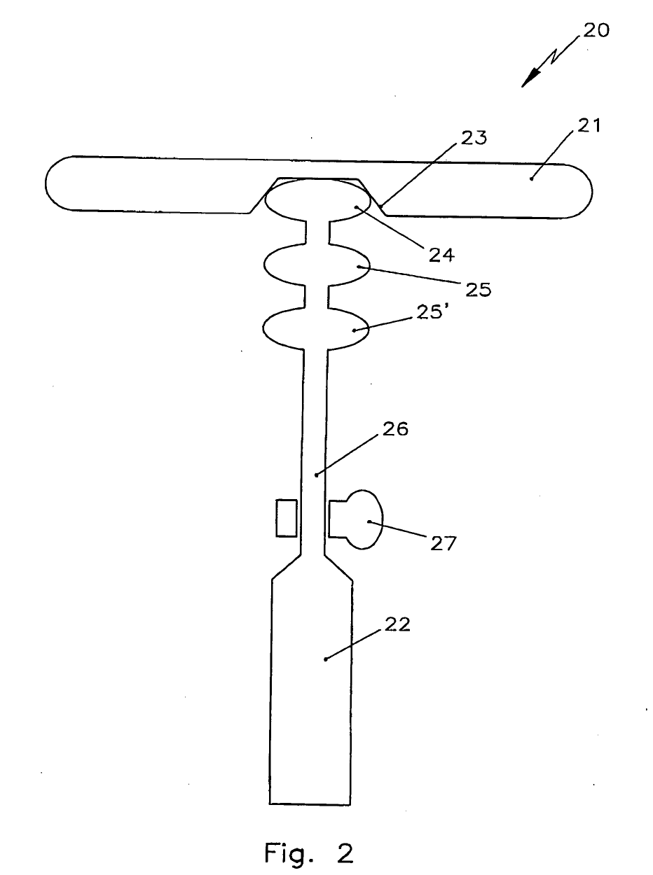

FIG. 2 is an embodiment with a second fastening element which is punch-shaped instead of bell-shaped, and has several ellipsoidal rotary elements on the shank; and

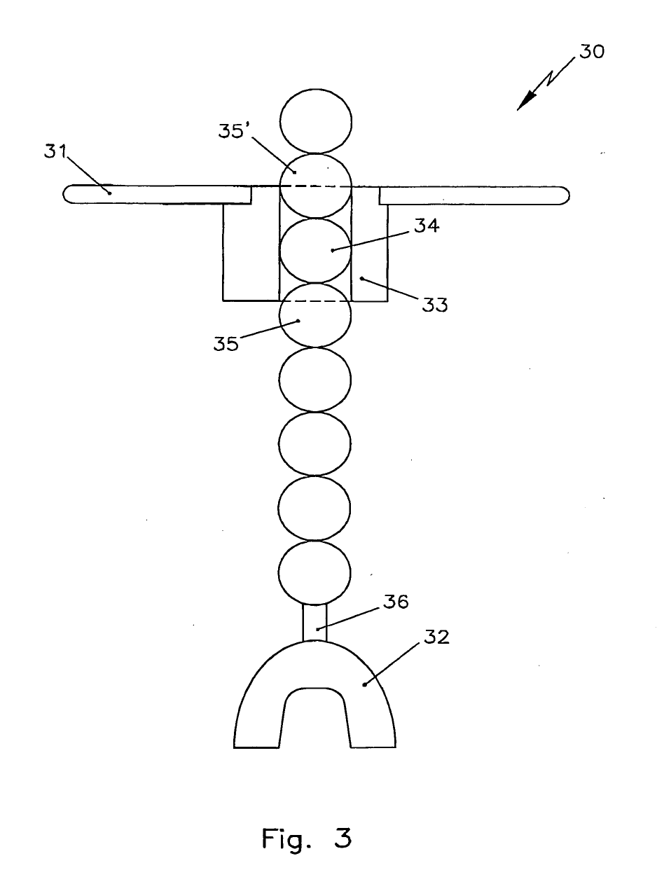

FIG. 3 shows a further embodiment with a chain of spherical rotary elements on the shank and shows how they pass through the receiving part.

Ossicle prosthesis 10 depicted highly schematically in FIG. 1 in a longitudinal cross section includes a first fastening element 11, which is designed as a top plate which rests on the tympanic membrane. A second fastening element 12 is provided on the other end of ossicle prosthesis 10, which has a bell shape in the present exemplary embodiment, and serves to fasten ossicle prosthesis 10 to a component of the ossicular chain, e.g., to the incus or the stapes.

First fastening element 11 is connected with second fastening element 12 via a longitudinal shank 16 which, on its end facing first fastening element 11, carries a rotary element 14 designed as a sphere, which is fixedly connected with shank 16. Rotary element 14 is enclosed by a cavity—designed as a cylindrical shell—of a receiving part 13 and is hingedly supported thereon.

Receiving part 13, which is made of an elastic plastic, is rigidly connected with first fastening element 11. In the embodiment shown in FIG. 1, it is connected by pressing a cylindrical section 18 located on the top side of receiving part 13 into a matching, circular bore 19 in the center of first fastening element 11. In addition to or as an alternative, cylindrical section 18 can be bonded or welded with circular bore 19. The elastic material of receiving part 13 is preferably selected such that its surface has good antifrictional properties at room temperature or body temperature. The elastic material is preferably silicone or PTFE, so that the mobility of rotary element 14 in receiving part 13 is particularly high due to the low surface friction drag.

Together with receiving part 13, rotary element 14 forms a rotary joint which provides inventive ossicle prosthesis 10 with post-operative mobility and good sound conduction properties due to the rigid, relatively hard connection of the rotary joint with the two fastening elements 11, 12.

These properties also exist with the other embodiments of the present invention, one of which is shown in FIG. 2. In this case as well, first fastening element 21 of ossicle prosthesis 20 is designed as a top plate to be placed on the tympanic membrane. In this case, second fastening element 22 is designed as a plunger for coupling ossicle prosthesis 20 directly to the inner ear. In this embodiment, receiving part 23 is a component of the top plate, and its cavity, which encloses a rotary element 24 designed as an ellipsoid of revolution on the end of its longitudional shank 26, is formed by a conical bore in the underside of first fastening element 21.

In the embodiment depicted in FIG. 2, a large number of further, adjoining rotary elements 25, 25′—only two of which are depicted in the drawing—are provided along the axis of longitudinal shank 26, which also fixedly connects second fastening element 22 with rotary element 24. To adjust the desired end position of longitudinal shank 26, it is cut off before rotary element 24 is installed in cavity of receiving part 23 on the top side of rotary element 24, e.g., using pincers or another suitable microinstrument. After assembly, receiving part 23 and rotary element 24 which has been snapped into its cavity together form a ball joint, and further rotary elements 25, 25′ no longer have a function. In terms of manufacture, it is favorable when the shape of further rotary elements 25, 25′ is identical to that of rotary element 24—they have the same outer diameter in particular—and they are located equidistantly along the axis of shank 26.

The mass distribution of the individual parts of the inventive prosthesis can be calculated depending on a desired, specifiable response of sound conduction in the middle ear, to allow the sound propagation properties to be tuned in an individualized manner.

In the embodiment in FIG. 2, an additional mass 27 is depicted schematically; it can be fastened, e.g., via a clip, at a suitable point on ossicle prosthesis 20, preferably on longitudinal shank 26. The size of mass 27 and its exact fastening position on the prosthesis are selected depending on a desired, specifiable response of sound conduction in the middle ear.

Finally, FIG. 3 is a depiction of an ossicle prosthesis 30, with which longitudinal shank 36 also includes a large number of further, adjoining rotary elements 35, 35′, and which differs from the other embodiments of the present invention shown in-that rotary elements 34, 35, 35′ located equidistantly along the axis of shank 36 are designed as balls of the same size. To shorten the length, these balls can be extended through a through-hole in receiving part 33 in the direction toward the bearing side of top plate 31 on the tympanic membrane, and the balls which extend past further rotary element 35′ can be simply cut off. In this embodiment, shank 36 ends in a bell-shaped, second fastening element 32.

In further embodiments of the inventive ossicle prosthesis which are not depicted separately in the drawing, the receiving part and the associated rotary element can also have other geometries, so that, together, they can form a rotary joint in the installed state. For example, the rotary element can be formed by a hemisphere which is fixedly connected with the longitudinal shank, and the cavity of the receiving part can be cylindrical, so that it exerts a small amount of pressure on the rotary element when the prosthesis is installed.

What is claimed is:

1. An ossicle prothesis (10; 20; 30) which replaces or bridges at least one component or parts of a component of the ossicular chain, in the case of which the ossicle prosthesis (10; 20; 30) includes, at one end, a first fastening element (11; 21; 31) designed as a top plate for mechanical connection with the tympanic membrane and, at the other end, a second fastening element (12; 22; 32) for mechanical connection with a component or parts of the ossicular chain or with the inner ear; it also includes a rotary joint between the two fastening elements (11, 12; 21, 22; 31, 32), which includes a receiving part (13; 23; 33) in which a rotary element (14; 24; 34)—which is fixedly connected with a longitudinal shank (16; 26; 36) which connects the two fastening elements (11, 12; 21, 22; 31, 32) with each other—is hingedly supported, wherein
the receiving part (13; 23; 33) is made of an elastic plastic, the receiving part (13; 23; 33) is rigidly connected with the first fastening element (11; 21; 31), and the receiving part (13; 23; 33) includes a cavity which is shaped such that, in the installed state, it encloses at least half of the surface of the rotary element (14; 24; 34).

2. The ossicle prosthesis as recited in claim 1, wherein
the elastic material of the receiving part (13; 23; 33) is selected such that its surface has good antifrictional properties at room temperature and body temperature.

3. The ossicle prosthesis as recited in claim 1, wherein
the rotary element (14; 34) is designed as a sphere or part of a sphere, particularly as a hemisphere.

4. The ossicle prosthesis as recited in claim 1, wherein
the rotary element (24) is designed as an ellipsoid of revolution or part of an ellipsoid of revolution.

5. The ossicle prosthesis as recited in claim 3, wherein
the cavity of the receiving part (13) has the shape of a spherical shell.

6. The ossicle prosthesis as recited in claim 1, wherein
the rotary element (24) engages in a cavity formed as a conical opening of the receiving part (23).

7. The ossicle prosthesis as recited in claim 1, wherein
the cavity of the receiving part (33) is cylindrical and, when the prosthesis is installed, the cavity presses slightly on the rotary element (34).

8. The ossicles prosthesis as recited in claim 1, wherein
the receiving part (13; 33) is inserted into and, particulary, is pressed into the first fastening element (11; 31).

9. The ossicles prosthesis as recited in claim 1, wherein
the receiving part (13; 33) is bonded or welded together with the first fastening element (11; 31).

10. The ossicles prosthesis as recited in claim 1, wherein
the receiving part (23) is a component of the first fastening element (21), which is designed as a top plate.

11. The ossicle prosthesis as recited in claim 1, wherein
the receiving part (13; 23; 33) is made of silicone or PTFE.

12. The ossicle prosthesis as recited in claim 1, wherein
the rotary element (14; 24; 34) and the longitudinal shank (16; 26; 36) are made of metal.

13. The ossicles prosthesis as recited in claim 1, wherein
the longitudinal shank (26; 36) includes a large number of further rotary elements (25, 25′; 35, 35′) which abut each other.

14. The ossicles prosthesis as recited in in claim 13, wherein
the shape of the adjoining, further rotary elements (25, 25′; 35, 35′) of the longitudinal shank (26; 36) is identical to that of the rotary element (24; 34)—they have the same outer diameter in particular—and they are located equidistantly along the axis of the shank (26; 36).

15. The ossicle prosthesis as recited in claim 1, wherein
the second fastening element (12; 32) is designed in the shape of a plate, a bell, a sleeve or a punch, or it is designed as a clip.

16. The ossicle prosthesis as recited in claim 15, wherein
the ossicle prosthesis (10; 30) is fastened to the tympanic membrane at one end and to the incus or stapes at the other.

17. The ossicle prosthesis as recited in claim 1, wherein
the ossicles prosthesis (20) is coupled via one end directly to the inner ear—using a plunger (22), in particular—by opening up the human cochlea (=cochleotomy).

18. The ossicle prosthesis as recited in claim 1, wherein
the entire ossicle prosthesis (10; 20; 30) or parts thereof are made of biocompatible plastics, particularly silicone, and/or of fibrous composite materials.

19. The ossicle prosthesis as recited in claim 1, wherein
parts of the ossicle prosthesis (10; 20; 30) are made of titanium and/or gold and/or tantalum and/or an alloy of these metals.

20. The ossicle prosthesis as recited in claim 1, wherein
parts of the ossicle prosthesis (10; 20; 30) are made of a material with memory effect, Nitinol in particular.

21. The ossicles prosthesis as recited in claim 1, wherein
the mass distribution of the individual parts of the prosthesis is calculated depending on a desired, specifiable frequency response of sound conduction in the middle ear.

22. The ossicles prosthesis as recited in claim 1, wherein
at least one additional mass (27), which depends on a desired, specifiable frequency response of sound conduction in the middle ear, is fastened to the ossicles prosthesis (20) or to part of the ossicular chain, using a clip in particular.

23. The ossicle prosthesis as recited in claim 1, wherein
the prosthesis is connected with an active vibrating part of a hearing aid which is active and implantable, in particular.
